(19) United States
(12) Patent Application Publication
(10) Pub. No.: US 2011/0262923 A1
(43) Pub. Date: Oct. 27, 2011

(54) METHOD FOR THE QUANTITATIVE DETERMINATION OF THE NUMBER OF COPIES OF A PREDETERMINED SEQUENCE IN A SAMPLE

(75) Inventor: Wolfgang Mann, Neudrossenfeld (DE)

(73) Assignee: Beckman Coulter, Inc., Brea, CA (US)

(21) Appl. No.: 13/124,372

(22) PCT Filed: Apr. 15, 2009
(86) PCT No.: PCT/EP2009/002759

§ 371 Date: Jul. 11, 2011

(30) Foreign Application Priority Data
Apr. 16, 2008 (DE) 10 2008 019 132.9

(51) Int. Cl. C12Q 1/68; G01N 27/447; G01N 33/559

(52) U.S. Cl. 435/6.12; 435/6.1; 204/456

(57) ABSTRACT

A method for the quantitative determination of the number of at least one predetermined sequence in a biological sample comprises the steps:
a) providing a biological sample containing a nucleic acid,
b) fragmenting the nucleic acid contained in the biological sample,
c) dividing the sample obtained in the step b) into y subsamples,
d) adding at least two primer pairs to each of the at least two subsamples, where to each of the subsamples the same primer pairs are added, and where the individual primer pairs are adapted to amplify, in an amplification reaction, subsequences of the predetermined sequence that are different for each primer pair,
e) carrying out an amplification reaction with each of the at least two subsamples obtained in the step d),
f) determining the number of different amplification products obtained with the amplification reactions in the step e) for the individual sub samples and the determination of the number of subsamples in which identical amplifications products have been obtained.

The present invention relates to a method for the quantitative determination of the number of at least one predetermined sequence in a biological sample, preferably in a single cell, and in particular to a method for the determination of the absolute number of copies of alleles per cell.

Methods for the quantification of sequences, in particular for the quantitative determination of the number of copies of nucleic acid sequences per cell, are gaining an increasingly more important role in molecular diagnostics. Since numerous diseases, some serious, are caused by deviations from the normal number of copies of nucleic acid sequences in the genome, corresponding diseases can be diagnosed reliably even in the early stages of their development by a determination of the number of copies of certain chromosomes or certain gene segments.

Examples of, in some cases serious, anomalies that are to be traced back to an increased number of copies of entire chromosomes are trisomy 18 (Edwards syndrome), trisomy 13 (Patau syndrome), and trisomy 21 (Down syndrome). In each of these diseases the number of copies of the corresponding chromosome 18, 13, or 21 per cell is three, whereas healthy individuals have only two copies of the above-mentioned chromosomes per cell. In all three cases the increase in the number of copies of the chromosomes in question leads to the most serious developmental disturbances. While carriers of trisomy 21 are drastically inhibited in their development and have deformities that are serious in some cases, carriers of trisomy 18 and 13 usually die within the first year of life.

Along with diseases that are to be traced back to an increased number of entire chromosomes, numerous diseases are known that are based on a modified number of copies of genes of gene segments.

The cause of Huntington's disease, a progressive neurodegenerative disorder characterized by abnormal, involuntary movements with increasing decline of cognitive and physical faculties, is supposed to be the tandem repetition of more than 37 copies of a certain motif (CAG), where the predisposition to develop the disease increases with the number of repetitions of this motif. Additional examples of unstable trinucleotide sequences in humans are Kennedy's disease and spinocerebral ataxia-1.

In addition it is known that certain proto-oncogenes can multiply by gene amplification in the genome. Amplifications of this type are often to be seen in the chromosome set as so-called “double minutes” (DM) or as “homogeneously staining regions” (HSR). Due to the enormous increase of the number of copies of the gene the corresponding protein is produced in the cells in very large amounts, which enable an enhanced activation of cell proliferation without a change of the individual gene per se. In particular, the myc proto-oncogene is supposed to be particularly affected by amplification.

Due to the need for methods for quantification of sequence copies in a biological sample numerous corresponding methods have been proposed in the past.

One of the fundamental quantification methods that provides at least information concerning the presence or absence of nucleic acid sequences and, depending on the process management, also a tentative conclusion regarding the number of copies of the relevant nucleic acid sequence per cell, is the so-called FISH method (fluorescence in situ hybridization). In this method the biological sample to be investigated, after appropriate pretreatment, i.e., denaturing with formamide and prehybridization with one or more different probes that have been marked previously with fluorescent dyes that are different for each probe, is incubated under conditions which enable a hybridization of the probes with sequences in the biological sample that are homologous to said probes. After the hybridization the samples are washed, whereby non-specific hybridization signals are eliminated. Subsequently the fluorescence signals of the preparation are evaluated with a fluorescence microscope. Each fluorescence signal present indicates the presence of the sequence corresponding to the probe provided with the corresponding fluorescence marker. The intensity of the fluorescence can permit a tentative conclusion regarding the number of sequence copies in the biological sample. If on the contrary at the wavelength of one of the fluorescence-marked probes used no signal is obtained, or only a signal lying below a defined threshold value is obtained, it can be inferred that the sequence corresponding to the corresponding probe is absent in the biological sample. However, the absence of a corresponding fluorescence signal can also be based on the fact that in the corresponding binding site of the sequence to be detected a mutation and/or microdeletion has taken place, due to which, under the hybridization conditions selected, the probe no longer binds to the predetermined sequence. An additional disadvantage of the method mentioned above lies in the fact that an undesired cross-hybridization leading to incorrect results can never be entirely ruled out. In addition this method is comparatively expensive, for one thing because fluorescence dyes strictly must be used and for another thing because expensive apparatus such as fluorescence microscopes are needed. Finally, the informative power of this method depends to a quite significant extent on the quality of the probes used and reliable results are only obtained when the probes hybridize at an effective rate of more than 90% at their corresponding binding sites so that 10% of the target sequences are not hybridized and consequently are no longer amplified. It follows from this that an incorrect choice of probes as well as inadequate hybridization conditions lead to an incorrect result. An additional disadvantage of this method lies in the fact that a minimum amount of biological sample must be used in order to obtain an evaluable fluorescence signal at all. In addition the sequence is not permitted to fall below a minimum length. Furthermore, it is necessary for an evaluable result to analyze a plurality of cells that were accessible to hybridization. For this reason the FISH analysis is not adequate for individual cell diagnostics. Moreover, an automated evaluation by the pathologist is hardly possible.

Another fluorescence-based method is CGH analysis (comparative genomic hybridization). In this method the nucleic acid of the sample to be analyzed is completely marked with a dye 1. The same amount of nucleic acids of a reference sample is marked with a dye 2. Both reaction batches are hybridized together on a spread metaphase chromosome set, where the sequences obtained in both reaction batches compete for the binding sites on the spread chromosomes. Essentially, the ratio of dye 1 to dye 2 is set to 1:1 in all the hybridization sites. If the sample to be analyzed contains amplified regions (more than the usual number of copies of the reference), then dye 1 will predominate at this hybridization site. In case of a deletion in the sample to be investigated only dye 2 will be detected at this hybridization site. The reference measurement provides relative information concerning the frequency of sequences in the sample to be analyzed. However, this method is also complicated and expensive since absolute fluorescence intensities have to be measured. In addition it also requires the use of a specific, comparably high amount of starting material.

A special variant is array CGH, in which hybridization occurs not on chromosomes but rather on immobilized sequences whose physical address in the genome is known.

An additional known method for the quantification of nucleic acid sequences is the real-time method in which a PCR (polymerase chain reaction) is carried out with fluorescence-marked primers and the increase of the fluorescence signal as a function of the number of cycles is observed. The threshold value PCR cycle (also threshold cycle) is assigned to the reaction time at which the fluorescence signal rises significantly from the background fluorescence and the PCR product formation increases exponentially. This correlates with the initial number of copies of the DNA sequence to be multiplied. In this way DNA samples can be quantified relatively by comparison to a DNA dilution series. A disadvantage of this method lies in the fact that the starting material cannot be scaled down in an arbitrary manner, since with only a few starting molecules, e.g., 10 to 100 copies, as starting material the stochastic error due to the exponential amplification becomes very large, which makes it no longer possible to provide quantitative information. Furthermore, this method also requires complicated and expensive apparatus for the measurement of the fluorescence intensity.

A newer method for the quantitative determination of a nucleic acid sequence is QF-PCR (quantitative fluorescence PCR), in which, in one PCR batch, several PCRs are carried out in parallel using different fluorescence-marked primers and the fluorescence-marked PCR products are subsequently analyzed by laser densitometry using an automatic DNA scanner. In order to be able to make a strongly informative quantitative comparison between PCR products amplified next to one another the two PCR partial reactions must run with equal efficiency and the fluorescence intensities of the reaction products at the time of the exponential product amplification must be analyzed quantitatively.

Other methods based on probes marked so as to be optically active, e.g., those using infrared-marked probes, also do not solve the problem.

A method based on QF-PCR methodology for the determination of possible spatial aberrations in chromosomes 21, 18, 13, X, and Y in amniotic fluid samples has been described by Lucchini et al. in Wissenschaftliche Informationen [Scientific Papers], September 2004. This method is based on in-vitro PCR amplification of repetitive and polymorphic STR (short tandem repeats) sequences with fluorescence-marked primers. After completion of the PCR the amplified PCR products are quantified by means of capillary electrophoresis. If chromosome-specific STR systems are used in this method, then from the number of the different PCR products obtained conclusions regarding the number of copies of the chromosome can be drawn. If, for example, in the reaction with a chromosome-specific STR system three peaks are obtained in the capillary electrophoresis, where the peak heights are in a 1:1:1 ratio to one another, then the investigated individual has three different alleles of the corresponding chromosome (triallelic trisomy). If on the contrary two peaks are obtained in the method, where the ratio of the peaks to one another is 2:1, then the investigated individual has per cell two identical alleles of the chromosome and another allele of the chromosome (diallelic trisomy). In the case that only two peaks with identical peak heights are obtained, the individual has two alleles so that there is no trisomy (heterozygous case). However this method provides no information concerning the presence or absence of a trisomy in the case that only one peak is obtained since this result is obtained in the case of a monoallelic trisomy as well as in the case of a monoallelic disomy. A method based on this technology for detecting trisomy 13 has also been disclosed in DE 101 02 687 A1. In order to be able to distinguish between a monoallelic disomy and a monoallelic trisomy, it has been proposed in this method to amplify with the PCR three different STR DNA regions specific to chromosome 13. However, this method also has the disadvantage that fluorescence-marked primers have to be used. In addition, it requires the use of a minimum amount of DNA since otherwise the stochastic error due to the exponential amplification becomes very large and it is no longer possible to provide quantitative information with regard to diallelic trisomy. Finally, an additional disadvantage of the method mentioned above lies in the fact that it only works, with any reliability, in a narrow PCR window since only in this window are the peak heights proportional to the ratio of the starting material. Furthermore, this method also has the disadvantage that the absolute fluorescence intensity has to be determined.

All the methods mentioned above are based on the use of fluorescence-marked primers and require expensive apparatus for determining the fluorescence intensity. In addition it requires the use of a minimum amount of starting material in order to obtain at least passably reliable results.

In DE 10 2005 045 560 A1 it has been proposed, for the quantitative determination of the number of a predetermined sequence and, in certain cases, of the sequences in a biological sample that are homologous to the predetermined sequence, to carry out, with a defined amount of biological sample, at least one amplification reaction that is adapted to amplify several sequences that are not homologous to one another and that are comprised by the predetermined sequence before the number of the different amplification products obtained in the at least one amplification reaction is determined and the thus determined number is compared to at least one frequency distribution. In so doing, the frequency distribution is obtained by separately carrying out, under the same reaction conditions as those used for the biological sample to be investigated and repeatedly in each case, at least one amplification reaction with at least two different reference samples that each have a known number of copies of the predetermined sequence, that number of copies of the predetermined sequence being different from the number of copies of the predetermined sequence in each of the other reference samples, and subsequently determining the number of different amplification products that is obtained per reference sample. This method provides good results but requires a large number of PCR reactions to be carried out.

It is the object of the present invention to provide a method for the quantitative determination of one or more predetermined sequences in a biological sample, where said method can be carried out in a simple and economical manner, said method also and specifically provides reliable results for a small number of predetermined sequences present in the biological sample to be investigated, and said method can be carried out, in particular, with small amounts of starting material.

According to the invention this object is achieved by a method for the quantitative determination of the number of at least one predetermined sequence in a biological sample, in particular for the determination of the absolute number of copies of alleles per cell, said method comprising the following steps:
a) providing a biological sample containing a nucleic acid,
b) fragmenting the nucleic acid contained in the biological sample,
c) dividing the sample obtained in the step b) into at least two subsamples,
d) adding at least two primer pairs to each of the at least two subsamples, where to each of the subsamples the same primer pairs are added, and where the individual primer pairs are adapted to amplify, in an amplification reaction, subsequences of the at least one predetermined sequence that are different for each primer pair,
e) carrying out an amplification reaction with each of the at least two subsamples obtained in the step d),
f) determining the number of different amplification products obtained with the amplification reactions in the step e) for the individual subsamples and determining the number of subsamples in which identical amplification products have been obtained.

The term fragmentation in the sense of the present invention is understood to mean the splitting of nucleic acid molecules into at least two nucleic acid molecules. The nucleic acid fragments arising from each nucleic acid molecule through fragmentation accordingly have a shorter molecular length than the starting molecules.

Furthermore, the term predetermined sequence in the sense of the present invention is understood to mean any sequence that is comprised by the nucleic acid contained in the biological sample. In particular, the predetermined sequence can be a chromosome, a chromatid, a gene, or a gene segment.

According to a preferred form of embodiment of the present invention the nucleic acid contained in the sample prepared in the step a) is lysed between the method step a) and the method step b). This can, for example, be achieved by the addition of lysozyme to the sample, by heating the sample, or by the addition of denaturing agents such as, for example, urea, dithiothreitol (DTT), and the like to the sample.

Preferably in the method according to the invention each of the nucleic acid molecules contained in the biological sample is fragmented during fragmentation into at least five nucleic acid fragments, where the fragmentation can be accomplished, for example, by restriction hydrolysis, by shearing, by ultrasound, or by digestion with DNase. Along with this, the method parameters in the fragmentation, e.g., the type of restriction enzyme used or the frequency and duration of the ultrasound, can be set so that fragments with a desired length are obtained.

In contradistinction to the method according to the state of the art, in the method according to the invention the absolute fluorescence intensity of PCR products is not determined, as, for example, in quantitative PCR, QF-PCR, FISH, and CGH, and, as in the case of FISH and CGH, compared to the fluorescence intensity of a control sample or a reference sample but rather one determines merely the number of subsamples in which identical amplification products have been obtained. To that extent no fluorescence-marked primers have to be used in the method according to the invention. In so far as they are nonetheless used for the detection of the number of different PCR products obtained, the fluorescence intensity of the fluorescence-marked PCR products obtained does not have to be determined in a complicated manner but rather it merely has to be evaluated whether or not there is a fluorescence that in certain cases lies above a defined threshold value at a wavelength corresponding to one of the fluorescent dyes used. Thus the method according to the invention can be carried out in a simple and economical manner without costly apparatus for the quantitative detection of fluorescence. An additional advantage of the method according to the invention is its rapid and simple executability because with it the number of amplification reactions that have to be carried out is low in comparison with the quantification method known from DE 10 2005 045 560 A1. The method according to the invention is suitable for the determination of the relative number of at least one predetermined sequence in a biological sample as well as for the determination of the absolute number of at least one predetermined sequence in a biological sample. For example, the method according to the invention can be used to determine a trisomy in the framework of an in vitro fertilization (IVF) or in the framework of analysis of fetal cells from maternal blood.

The method according to the invention is suitable in particular also for the quantitative determination of the number of at least one predetermined sequence in a biological sample that contains only a small amount of nucleic acid, for example, only a few cells. Preferably the method according to the invention is carried out with a biological sample that contains as starting material only one cell, for example, a human cell such as, for example, a polar body, an animal cell, or a plant cell, or alternatively with an individual cell held in suspension.

The fundamental principle of the method according to the invention is based on the determination of the number of copies of a predetermined nucleic acid sequence, such as, for example, the number of copies of a chromosome, by fragmenting the nucleic acid, dividing the fragmented sample into several subsamples, and carrying out a multiplex PCR with each of the subsamples, where in each of the multiplex PCR reactions carried out with the individual subsamples the same primer pairs are used, before one subsequently determines the number of subsamples in which identical amplification products have been obtained. From this comparison the number of copies of the predetermined sequence can then be determined with the required certainty. Let this be explained in the following Gedankenexperiment.

A cell may contain zero, one, or two copies of a predetermined sequence, for example, of chromosome 21. If the cell is lysed in the cell suspension and the sample thus obtained is divided into two subsamples, then in the case of monosomy, that is, if the cell contains one copy of chromosome 21, the chromosome 21 will pass to one of the two subsamples. Therefore in this case only one of the two subsamples contains chromosome 21. In contrast, in the case of disomy, i.e., if the cell contains two chromosomes 21, each of the subsamples can contain a copy of chromosome 21 or one of the subsamples can contain no chromosome 21 and the other subsample can contain two copies of chromosome 21. If the sample contains no copy of chromosome 21, then naturally it is not found in either of the two subsamples.

If the sequence to be determined, i.e., chromosome 21, is divided by fragmentation into several subsequences, e.g., by restriction hydrolysis into 10 different partial sequences, the probability that, in the case of disomy, at least one pair of the identical subsequences passes to both subsamples is nearly 100%, whereas in the case of monosomy once again each subsequence can be present in at most one of the two subsamples. In case of nullisomy a subsequence of chromosome 21 will naturally not be in either of the two subsamples. If the method steps d) to f) of the method according to the invention are carried out, in the case of monosomy each subsequence can only be detected in one of the two subsamples, whereas in the case of disomy at least some of the subsequences of chromosome 21 will be present in both subsamples and in the case of nullisomy a subsequence will not be present in any of the subsamples. Therefore it can be decided unequivocally whether the sample is null, or contains one or two copies of chromosome 21 by dividing the fragmented sample into two subsamples and the subsequent carrying out of the method steps d) to f), with a sufficiently large number of primer pairs used during the method step d) and to the extent that during the method steps a) to c) processing is under non-denaturing conditions, i.e., the DNA is present in double-strand form during those methods steps. Through an increase of the number of subsamples into which the fragmented sample is divided in the method step c) nullisomy, monosomy, disomy, trisomy, etc. can be distinguished from one another and in fact in the case that the DNA is denatured before the division into the subsamples, i.e., is converted into single strands.

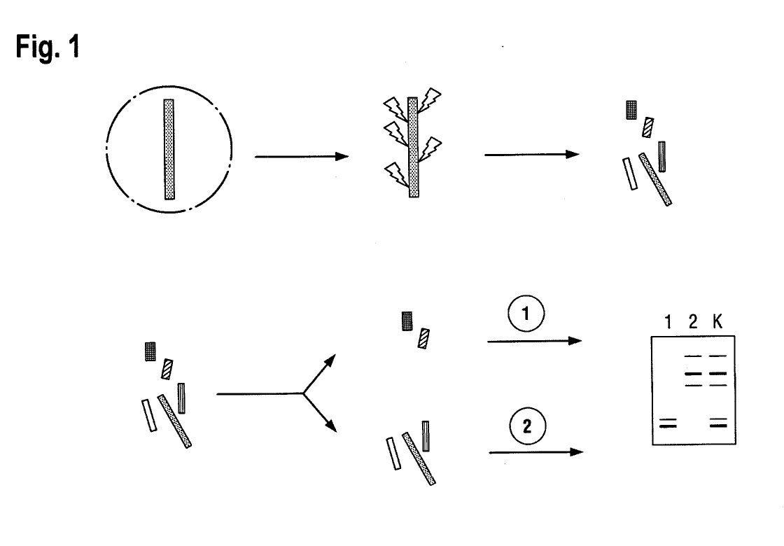

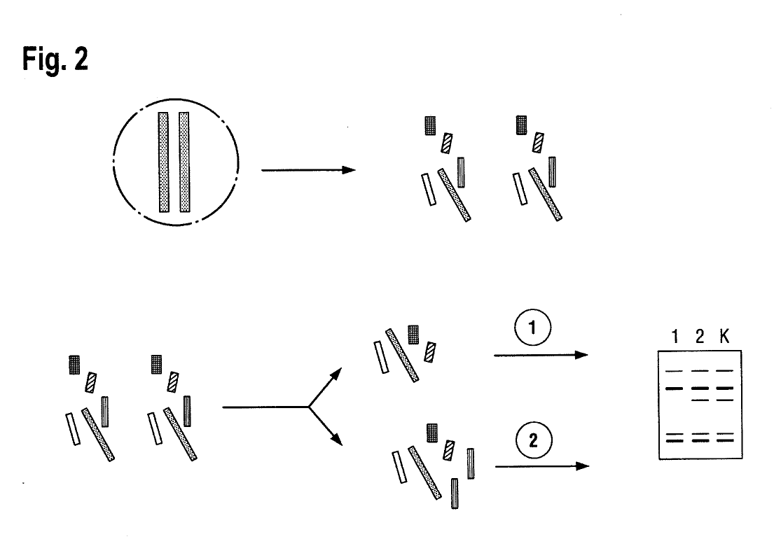

The Gedankenexperiment above is represented schematically in the case of monosomy and disomy in FIGS. 1 and 2.

In FIG. 1 an individual cell symbolized in FIG. 1 at the upper left by a circle is used in the method according to the invention, said single cell containing a copy of the sequence to be quantified, in this case chromosome 21, which is symbolized by the gray bar contained in the circle. This single cell is lysed in suspension (not represented) before a restriction enzyme is added to the suspension, said restriction enzyme fragmenting chromosome 21 contained in the sample, as represented in the center and upper right of FIG. 1, into five subsequences, each of a different length. Subsequently the sample containing these five subsequences, as represented in FIG. 1 at the lower left and in the center, is divided into two subsamples, where each of the five subsequences passes, with a probability of 50%, to one of the two subsamples. In the case represented in FIG. 1 the two short subsequences are contained in the subsample 1 while the three longer subsequences are contained in the subsample 2. To each of the two subsamples 5 identical primer pairs are subsequently added, where each of the five primer pairs is specific for one of the subsequences of chromosome 21 that were previously produced by fragmentation. In connection with this the 5 primer pairs in the case represented in FIG. 1 are conceived so that each of the primer pairs produces in the following PCR a PCR product of different length. Then a PCR is carried out under identical conditions for each of the two subsamples as well as with a control sample that contains chromosome 21 and subsequently a subsample of all three PCR batches is applied to a polyacrylamide gel and separated electrophoretically. As is to be inferred from the gel represented schematically in FIG. 1 at the lower right, 5 different bands are obtained for the control sample (K), only 2 bands are obtained for the subsample 1 (1), only 3 bands are obtained for the subsample 2 (2), and none of the bands obtained for subsample 1 have the same length as the bands obtained for the subsample 2. From this it can be concluded with a high probability that exactly one copy of chromosome 21 was contained in the sample. By increasing the number primer pairs used in the PCR reactions from 5 to, for example, 10 the statistical certainty of the results obtained can be increased correspondingly.

In FIG. 2 the same method is reproduced schematically for the case of a biological sample that contains two chromosomes 21 (disomy). In this case two copies of each of the five subsequences of chromosome 21 are obtained in the fragmentation. Each of these subsequences is passed, with a probability of 50%-, to one of the two subsamples so that the probability of the two copies of a subsequence being in one subsample is 50% and the probability that one of the two copies of a subsequence is in a subsample while the other copy of the same subsequence is in the other subsample is also 50%. The probability that at least for one of the five different subsequences the two copies of the subsequence pass to two different subsamples is therefore more than 95%. In the case represented in FIG. 2, of the two copies of four of the subsequences one copy is contained in subsample 1 and one copy is contained in the subsample 2, whereas the two copies of the fifth subsequence are contained only in subsample 2. After carrying out the PCR reactions it accordingly follows in the gel-electrophoretic analysis that for the subsample 1 (1) four bands are obtained, whereas for the subsample 2 (2) and for the control (K) five bands are obtained. Therefore the subsamples 1 and 2 each have four identical PCR products, from which it follows that the sample contained at least two chromosomes 21. The cases of monosomy and nullisomy can however be ruled out based on the results. By increasing the number of subsamples into which the biological sample is divided in the method step c) the absolute number of copies of chromosome 21 in the sample could also be determined, that is, it can be determined whether chromosome 21 is present in the sample in 2, 3, 4, 5, etc. copies.

In principle the method steps a) to c) of the method according to the invention are carried out under non-denaturing conditions so that the predetermined sequence, to the extent it is double-stranded DNA, is passed in the form of double-stranded nucleic acid to the individual subsamples. Alternatively to this it is however also possible to denature the nucleic acid in the biological sample before, as occurs in the method step c), the division of the fragmented sample into at least two subsamples in order to convert double-stranded nucleic acid that is present into single-stranded nucleic acid because single-stranded nucleic acid, similarly to double-stranded nucleic acid, can lead to a positive PCR result. This denaturing can, for example, be carried out before or after the fragmentation according to the method step b). In this case in the method step c) there are accordingly two times more copies of the fragmented predetermined sequence that are to be passed to the subsamples than there were in the initial biological sample, namely in the case of a disomy, for example, four single-stranded copies of the fragmented predetermined sequence, whereas in the initial biological sample there were two double-stranded copies of the unfragmented predetermined sequence. Also in the case that the predetermined sequence is already present in the initial sample as a single strand, e.g., as RNA, a denaturing can be done before carrying out the method step c), for example, in order to break up secondary structures of the single-stranded nucleic acids before the fragmentation occurring enzymatically.

With regard to the type of primer pairs used the method according to the invention is not limited so that in principle specific as well as non-specific primer pairs can be used. However, the use of specific primer pairs is preferred because the length of the PCR products can be set selectively and it is ensured that the genetic subsequences are amplified.

In an extension of the inventive concept it is proposed to adapt the at least two primer pairs added in the method step d) to amplify in an amplification reaction subsequences of the at least one predetermined sequence that are each different and do not overlap.

According to an additional preferred form of embodiment of the present invention it is provided to carry out the fragmentation in the method step b) in such a manner and/or to select the primer pairs added in the method step d) in such a manner that the average length of the nucleic acid fragments obtained in the method step b) is greater than the length of the subsequences of the at least one predetermined sequence that are amplified with the primer pairs added in the step d).

To the extent that the fragmentation is done by restriction hydrolysis the restriction enzyme or the restriction enzymes is/are preferably chosen so that they do not cleave in the sequence regions to be amplified. This can, for example, be ensured by the fact that first of all in the nucleic acid comparatively rarely cleaving restriction enzymes are selected and then depending on the actual cleaving sites the primers are selected so that the cleaving sites of the restriction enzymes do not lie in the regions included in the primer binding sites.

It is a significant method step of the method according to the invention that after carrying out the amplification reactions with the individual subsamples there is a determination of the number of subsamples in which identical amplification products have been obtained, in order to determine from that the number of the predetermined sequence in the biological sample. This determination of the number of subsamples in which identical amplification products have been obtained naturally assumes that the various amplification products are different from one another, that is, the number of different amplification products obtained per subsample is determined. This is preferably done by electrophoresis, e.g., by gel electrophoresis such as polyacrylamide electrophoresis or agarose gel electrophoresis, or by capillary electrophoresis. Alternatively thereto a fluorometric determination of the amplification products is also possible.

In order in this form of embodiment of the present invention to alleviate the expenditure of energy in the electrophoresis, in particular when polyacrylamide electrophoresis or agarose gel electrophoresis used, and in order to reduce the duration of the electrophoresis it is proposed in an extension of the inventive concept to select the primer pairs added in the method step d) in such a manner that all the different subsequences that can be amplified with the individual primer pairs differ in their length by in each case at least 10 base pairs, preferably by at least 25 base pairs, particularly preferably by at least 50 base pairs, and quite particularly preferably by at least 100 base pairs. In this way no special demands are made on separation power in the gel electrophoresis.

In order to enhance the reliability of the results of the method according to the invention it is provided according to an additional preferred form of embodiment of the present invention furthermore to carry out a method step g₁) that includes the comparison of the amplification products determined in the method step f) for each subsample with the amplification products obtained in an amplification reaction with at least one control sample, where the amplification reaction carried out with the at least one control sample is carried out with the same primer pairs as in the step d) and the control sample preferably contains a known number of copies of the predetermined sequence. In this way any faults in carrying out the amplification reaction, such as, for example, a temporary failure of the thermocycler during the amplification reaction, can be recognized with the aid of the comparison of the band patterns obtained with the control sample with the band patterns expected for the control sample. Thus, for example, it is possible to validate the case of nullisomy, in which no bands are expected for the amplification reactions carried out with the fragmented subsamples of the sample, because if the expected bands are obtained for the control sample a fault in carrying out the amplification reaction can be ruled out.

For this reason the amplification reaction with the at least one control sample is preferably carried out in parallel to the step e).

Alternatively to the form of embodiment mentioned above the method according to the invention can include instead of the method step g₁) also the additional method step g₂) that includes a comparison of the amplification products determined in the step f) for each subsample with a data set where the data set includes data relating to the different amplification products obtainable with at least one control sample in an amplification reaction using the primer pairs added in the step d).

In principle, it is the case that the statistical certainty of the results obtained with the method according to the invention is greater the more primer pairs are used in the amplification reaction, preferably a multiplex PCR. However, the experimental complexity increases as the number of primer pairs used increases. As a compromise between these two tendencies running counter to one another it is preferred in the method step d) to add to each of the subsamples, per predetermined sequence to be determined, 2 to 50, preferably 5 to 25, particularly preferably 8 to 15, quite particularly preferably 10 to 15, and most preferably 12 primer pairs.

Also for the number of subsamples prepared in the method step c) it is the case that in particular for larger numbers of copies the statistical certainty of the results obtained with the method according to the invention is greater the more subsamples the fragmented sample is divided into in the method step c) whereas the experimental complexity increases as the number of subsamples prepared in the method step c) increases. As a compromise between these two tendencies running counter to one another it is preferred to divide the fragmented sample in the method step c) into at least 3 subsamples, preferably into at least 4 subsamples and particularly preferably into 5 to 20 subsamples. Preferably the sample obtained in the method step b) is divided in the method step c) so that the at least two subsamples produced have the same volume. Apart from the fact that this simplifies carrying out the method step c), it also simplifies later calculations of the results.

In order to keep the amount of apparatus and manual labor as small as possible, it is proposed in an extension of the inventive concept to carry out the amplification reactions in the method step e) for all the subsamples in parallel on a substrate consisting of, for example, glass, where the individual subsamples or subsamples of these subsamples are each positioned on a reaction site of the substrate. In order to ensure a firm adhesion of the individual subsets or subsamples to the substrate, in particular even after coating with the successive oil drops, it is provided according to a further preferred form of embodiment of the present invention that each of the reaction sites on the substrate comprises a central hydrophilic area which on its outer side is encircled by a first hydrophobic area which in turn on its outer side is encircled by a central hydrophilic area which on its outer side is encircled by a second hydrophobic area.

Good results are obtained with this form of embodiment in particular when the central hydrophilic area is at least essentially circular and on its outer side is encircled concentrically by an at least essentially circular first hydrophobic area which in turn on its outer side is encircled concentrically by an at least essentially circular central hydrophilic area which in turn on its outer side is encircled by the second hydrophobic area.

Obviously, the necessary DNA polymerase and, in certain cases, at least one compound, selected from the group consisting of the pH buffers, salts, water, and additional customary PCR additives, for adjusting conditions suitable for carrying out the PCR are added to the sub samples of the fragmented sample before carrying out the amplification reactions according to the method step e).

With the method according to the invention the number of copies of a predetermined sequence contained in the biological sample or preferably the number of copies of several predetermined sequences are determined. Preferably the predetermined sequence or predetermined sequences are chromosomes so that with the method according to the invention in particular the number of copies of the 1 to 23, particularly preferably of 1 to 10 and quite particularly preferably of 1 to 5 chromosomes present in the biological sample can be determined.

According to a further preferred form of embodiment of the present invention the previously described method according to the invention for the quantitative determination of the number n of x predetermined sequence(s) in a biological sample includes the following steps:
a) providing a biological sample containing DNA,
b) fragmenting the DNA contained in the biological sample,
c) dividing the sample obtained in the step b) into y subsamples,
d) adding z primer pairs to each of the y subsamples, where to each of the subsamples the same primer pairs are added, and where the individual primer pairs are adapted to amplify, in a PCR, subsequences of the x predetermined sequence(s), said subsequences being different for each primer pair,
e) carrying out a PCR with each of the y subsamples obtained in the step d),
f) determining the number of different amplification products obtained with the PCR reactions in the step e) for the individual subsamples and determining the number of the z subsamples in which identical amplification products have been obtained.

Preferably x is a whole number between 1 and 23, y is a whole number greater than or equal to 4, preferably 5 to 20, and z is a whole number greater than or equal to 3·x, preferably 5·x to 25·x and particularly preferably 8·x to 15·x.

As presented in detail in particular in the Gedankenexperiment above, the number n of predetermined sequence(s) can be determined from the number of the z subsamples in which identical amplification products have been obtained. In the simple case of four subsamples (y=4) and 8 primer pairs used (z=8) the result means, e.g., when no denaturing of the DNA into single-strand DNA has been performed before the method step c), that identical amplification products have not been found in any of the subsamples but rather each of the individual amplification products occurs in only one subsample and that the number of copies of the predetermined sequence in the biological sample is 1 (monosomy). By contrast the number of copies is 0 (nullisomy) when no amplification product at all has been obtained in any of the subsamples. If on the contrary for at least two subsamples identical amplification products are obtained, the number of copies is at least two and so on.

Preferably the number of primer pairs used in the method step e) is selected so that the probability of a false negative result is at most 5%. This can, for example, be ensured by the statistical approach reproduced below:

By means of the method described below it is intended, for example, to provide information with regard to the number of copies of a certain chromosome that are present in a sample.

The probability of the correct determination of the number of copies here is a function of all of the following parameters:
k=copies actually present
N=number of partial reactions
(Multiplex degree=number of investigated bands of different length per partial reaction)
(Number of cells investigated).

The following statistical considerations clarify the determination of the probability of a correct determination of the number of copies:

1) Determination of all the possible combinations in the apportioning to the partial reactions of the sequence copies present:

Combinations with repetition A_{desired}=(N+k−1)!/(N−1)!*k!

In principle the following assumptions apply here:
a) All the elements (=partial reaction, for example, 1 to 8) are different from one another.
b) Several elements are selected (a partial reaction with a sequence copy is considered as “selected”).
c) An element can be selected repeatedly (that is, several sequence copies can be allocated to a specific partial reaction in the initial distribution).
   2) Determination of the possible combinations for the apportionment of the existing sequence copies to the partial reactions, where there is only one copy per partial reaction:

Combinations with repetition A_{in total}=N!/(N−k)!*k!

In principle the following assumptions apply here:
a) and b) as above.
c) An element is not allowed to be selected repeatedly (that is, at most one sequence can be allocated to a partial reaction in the initial distribution of the sequence copies).

If the ratio of the “desired” events (2) to all the possible events (1) is formed, then the percentage of the “desired” events is obtained and with that the probability of a correct inference from the method when using the corresponding parameters.

Probability P(correct inference)=A_{desired}/A_{in total}=(N−1)!*N!/(N+k−1)!*(N−k)!

Example
k=4
N=8
(Multiplex degree=number of investigated bands of different length per partial reaction)

Probability of the successful detection of all 4 sequence copies that are present.

=A_{desired}/A_{in total}=((8−1)!*8!)/((8+4−1)!*(8−4)!)=(7!*8!)/(11!*4!)=0.2121.

The probability of drawing an incorrect inference is thus 1−0.2121=0.7878. This applies when only one PCR product per subsample is analyzed.

However, if several PCR products per subsample are analyzed (multiplex-PCR), the total probability is obtained by multiplication of the individual probabilities. Therefore, for example, for a 3-plex: P(detection of all bands)=0.7878*0.7878*0.7878.

One arrives at the following table, depending on the multiplex degree.

Multiplex
degree

| | 1 | 2 | 3 | 4 | 5 | 6 |
|---|---|---|---|---|---|---|
| P(correct | 0.787 | 0.620 | 0.489 | 0.385 | 0.303 | 0.239 |
| result) | | | | | | |

| | Multiplex | | | | | | |
|---|---|---|---|---|---|---|---|
| | degree | | | | | | |
| | 7 | 8 | 9 | 10 | 11 | 12 | 13 |
| P(correct | 0.188 | 0.148 | 0.116 | 0.092 | 0.072 | 0.057 | 0.045 |
| result) | | | | | | | |

With a multiplex degree of 13 a probability of error of less than or equal to 5% is therefore obtained here.

However, since, for example, in the detection of chromosomes after their denaturing there are always 2n individual single strands, one can perform the statistical inference as follows.

P(successful detection of n chromosomes)=P(successful detection of exactly 2n chromatids)+P(successful detection of exactly (2n−1) chromatids). Thus from the detection of 1 or 2 amplificates one can infer the presence of a chromosome (=2 chromatids), from the detection of 3 or 4 amplificates one can infer the presence of 2 chromosomes (=4 chromatids), from the detection of 5 or 6 amplificates one can infer the presence of 3 chromosomes (=6 chromatids) and so on.

P(detection of exactly 2n chromatids)+P(detection of exactly (2n−1) chromatids)=

=(N−1)!*N!/(N+k−1)!*(N−k)!+(N!/k₀!*k₁!*k₂!* . . . *kₙ!)/((N+k−1)!/(N−1)!*k!)

where the following applies:

K₀=the number of reaction vessels in which no sequence copy is present,
K₁=the number of reaction vessels in which exactly 1 sequence copy is present,
K₂=the number of reaction vessels in which exactly 2 sequence copies are present and so on.

In the case of the detection of 2n−1 chromatids 2 sequence copies are present in one of the partial reactions and in all the others, if any at all, only 1 sequence copy is present: Thus the following applies here:

K₀=N−k+1
K₁=k−2

### K₂=1

K₃, . . . , kₙ=0

Therefore the formula reads:

P(detection of exactly 2n chromatids)+P(detection of exactly (2n−1) chromatids)=

=((N−1)!*N!)/((N+k−1)!*(N−k)!)+(N!/(N−k+1)!*(k−2)!)/((N+k−1)!/(N−1)!*k!)

Example
k=4
N=8

$$Probability of the successful detection of all 4 sequence copies that are present . = ( ( 8 - 1 ) ! * 8 ! ) / ( ( 8 + 4 - 1 ) ! * ( 8 - 4 ) ! ) + ( 8 ! / ( 8 - 4 + 1 ) ! * ( 4 - 2 ) ! ) / ( ( 8 + 4 - 1 ) ! / ( 8 - 1 ) ! * 4 ! ) = 0.212 + 0.509 = 0.721$$

The probability of drawing an incorrect inference is thus 1−0.721=0.278. This applies when only one PCR product per subsample is analyzed.

However, if several PCR products per subsample are analyzed (multiplex-PCR), the total probability is obtained by multiplication of the individual probabilities. Therefore, for example, for a 3-plex: P(detection of all bands)=0.278*0.278*0.278=0.021.

One arrives, independently of the multiplex degree at the following table.

| | Multiplex | | | | | |
|---|---|---|---|---|---|---|
| | degree | | | | | |
| | 1 | 2 | 3 | 4 | 5 | 6 |
| P(correct | 0.278 | 0.077 | 0.021 | 0.006 | 0.001 | 0.0004 |
| result) | | | | | | |

With a multiplex degree of 3 a probability of error of less than or equal to 5% is therefore obtained here.

One possible evaluation variant of the selective amplifications would be, for example, the detection by means of fluorescent dyes, where for each fragment of different size a respective fluorescent dye is selected. Since it is only of interest here whether products have been amplified while the amount of product on the contrary is not of interest, the fluorescence of the individual wavelengths could thus be measured simply with an endpoint determination without a gel having to be used.

Therefore the method according to the invention is suitable in particular for the quantitative determination of the number n of at least one predetermined sequence in a biological sample if the number n per predetermined sequence in the biological sample is 0 to 100, preferably 0 to 10, particularly preferably 0 to 5 and quite particularly preferably 0, 1, 2, 3, or 4.

For example, the method according to the invention can be carried out advantageously in the framework of an in vitro fertilization (IVF) or in the framework of analysis of fetal cells from maternal blood.

In the following the present invention is explained in more detail with the aid of an example, which is exemplary but does not restrict the present invention.

Example

With two biological samples, each containing cells and in the form of a suspension, the method according to the invention was carried out separately from one another in order to determine the number of copies of chromosome 21 that are present in the two samples. While the first sample contained two cells the second sample contained only one cell.

For this the two samples were each lysed and hydrolyzed with a mixture of the restriction enzymes Xho I, Sac I, and PacI at 37° C. for 5 minutes (Cek-Kit, Advalytix Products or Olympus Life Science Research Europa GmbH). Subsequently the fragmented samples obtained were each divided into four subsamples of equal volume, after which to each of the subsamples obtained 7 primer pairs were added. All seven primer pairs were specific for different partial sequences or subsequences of chromosome 21 that were different for each primer pair, where all seven primer pairs were selected so that each primer pair yields a PCR product of different length. These sample batches were each pipetted onto a reaction site of a glass substrate.

To each sample there was added 1 μl PCR mixture that contained 0.5 μm 2× Multiplex PCR Master Mix (Qiagen GmbH, Hilden), 0.005 μl bromophenol blue (0.1%), 0.06 μl 5× Q-Solution (Qiagen GmbH, Hilden), and 0.39 μl water. Subsequently the individual reaction batches were covered with 5 μl Sealing Solution (Advalytix AG, Munich) and subjected to a PCR with the following temperature profile:
10 minutes at 94° C.,
35 cycles with
   94° C. for 30 seconds,
   63° C. for 60 seconds,
   72° C. for 60 seconds, and
10 minutes at 72° C.,

Subsequently a subsample of each of the 8 PCR products was pipetted into a pocket of a polyacrylamide gel, separated electrophoretically, and detected after dyeing with silver.

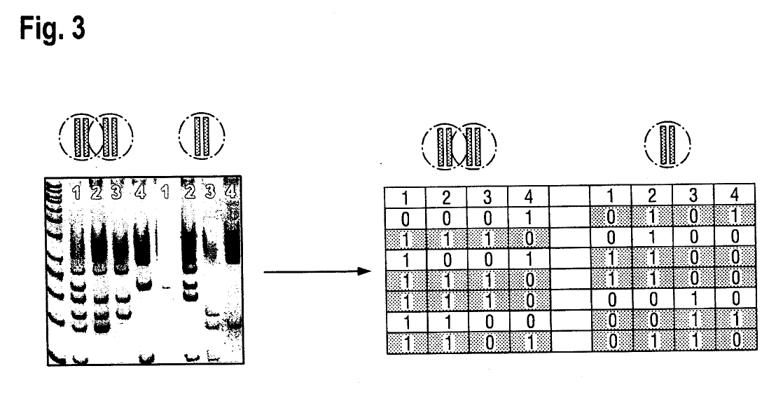

A photograph of the polyacrylamide gel is reproduced in FIG. 3. In FIG. 3 the numbers 1 to 4 identify the tracks of the individual subsamples of the PCR reactions for both probes, while in the unlabeled track on the left a length marker was applied. The result is summarized in the two tables reproduced in FIG. 3 on the right next to the photograph. The first line of the tables reproduces the individual tracks of the gel, that is, the tracks 1 to 4 corresponding to the subsamples 1 to 4 for the first sample and the tracks 1 to 4 corresponding to the subsamples 1 to 4 for the second sample. Among these and given in each column is the number of the bands for the seven possible PCR products that have been obtained for each individual subsample, that is, “0” or “1”.

As can be inferred from FIG. 3, for the first sample whose PCR products were applied for the individual subsamples in the left four tracks of the polyacrylamide gel, in at most three of the four subsamples identical PCR products were obtained, whereas for the second sample, whose PCR products were applied for the individual subsamples in the right four tracks of the polyacrylamide gel, in at most two of the four subsamples identical PCR products were obtained.

From this it follows that the first sample contained at least three copies of chromosome 21 whereas the second sample contained at least two copies of chromosome 21.

1. Method for the quantitative determination of the number of at least one predetermined sequence in a biological sample, in particular for the determination of the absolute number of copies of alleles per cell, said method comprising the steps:
a) providing a biological sample containing a nucleic acid,
b) fragmenting the nucleic acid contained in the biological sample,
c) dividing the sample obtained in the step b) into at least two subsamples,
d) adding at least two primer pairs to each of the at least two subsamples, where to each of the subsamples the same primer pairs are added, and where the individual primer pairs are adapted to amplify, in an amplification reaction, subsequences of the at least one predetermined sequence that are different for each primer pair,
e) carrying out an amplification reaction with each of the at least two subsamples obtained in the step d),
f) determining the number of different amplification products obtained with the amplification reactions in the step e) for the individual subsamples and determining of the number of subsamples in which identical amplification products have been obtained.

2. Method according to claim 1,
characterized in that
the at least one predetermined sequence is a chromosome, a chromatid, a gene, or a gene segment.

3. Method according to claim 1,
characterized in that
the biological sample is an individual cell or an individual cell held in suspension, where the individual cell is preferably a human cell, an animal cell, or a plant cell, particularly preferably, a polar body.

4. Method according to claim 1,
characterized in that
the fragmentation of the nucleic acid contained in the biological sample is carried out in the step b) by restriction hydrolysis, by shearing, by ultrasound, or by digestion with DNase.

5. Method according to claim 1,
characterized in that
the nucleic acid is double-stranded DNA and it is denatured after the step a) and before the step c) to form single strands.

6. Method according to claim 1,
characterized in that
the primer pairs added in the step d) are each specific for subsequences of the at least one predetermined sequence.

7. Method according to claim 1,
characterized in that
the at least two primer pairs added in the method step d) are adapted to amplify in an amplification reaction subsequences of the at least one predetermined sequence that are each different and do not overlap.

8. Method according to claim 1,
characterized in that
the fragmentation is carried out in the method step b) in such a manner and/or the primer pairs are selected in the method step d) in such a manner that the average length of the nucleic acid fragments obtained in the method step b) is greater than the length of the subsequences of the at least one predetermined sequence that can be amplified with the primer pairs added in the step d).

9. Method according to claim 1,
characterized in that
the primer pairs added in the method step d) are selected in such a manner that all the different subsequences that can be amplified with the individual primer pairs differ in their length in each case by at least 10 base pairs, preferably by at least 25 base pairs, particularly preferably by at least 50 base pairs, and quite particularly preferably by at least 100 base pairs.

10. Method according to claim 1,
characterized in that
the number, determined in the step f), of different amplification products obtained for the individual subsamples and/or the determination of the number of subsamples in which identical amplification products have been obtained is done by gel electrophoresis or by capillary electrophoresis.

11. Method according to claim 1,
characterized in that
it furthermore comprises the step g₁) of comparing the amplification products determined in the method step f) for each subsample with the amplification products obtained in an amplification reaction with at least one control sample, where the amplification reaction carried out with the at least one control sample is carried out with the same primer pairs as in the step d).

12. Method according to claim 11,
characterized in that
the amplification reaction with the at least one control sample is carried out in parallel to the step e).

13. Method according to claim 1,
characterized in that
it furthermore comprises the step g₂) of comparing the amplification products determined in the step f) for each subsample with a data set where the data set includes data relating to the different amplification products obtainable with at least one control sample in an amplification reaction using the primer pairs added in the step d).

14. Method according to claim 1,
characterized in that
in the step d) to each of the subsamples, per predetermined sequence to be determined, 2 to 50, preferably 5 to 25, particularly preferably 8 to 15, quite particularly preferably 10 to 15 and most preferably 12 primer pairs are added.

15. Method according to claim 1,
characterized in that
the sample obtained in the step b) is divided in the step c) into at least 3 subsamples, preferably into at least 4 subsamples and particularly preferably into 5 to 20 subsamples.

16. Method according to claim 1,
characterized in that
the amplification reactions in the method step e) are carried out for all the subsamples in parallel on a substrate consisting of, for example, glass, where the individual subsamples or subsamples of these subsamples are each positioned on a reaction site of the substrate.

17. Method according to claim 16,
characterized in that
each of the reaction sites on the substrate comprises a central hydrophilic area which on its outer side is encircled by a first hydrophobic area which in turn on its outer side is encircled by a central hydrophilic area which on its outer side is encircled by a second hydrophobic area.

18. Method according to claim 17,
characterized in that
the central hydrophilic area is at least essentially circular and on its outer side is encircled concentrically by an at least essentially circular first hydrophobic area which in turn on its outer side is encircled concentrically by an at least essentially circular central hydrophilic area which on its outer side is encircled by the second hydrophobic area.

19. Method according to claim 1,
characterized in that
the number of copies of the 1 to 23, particularly preferably 1 to 10, and quite preferably 1 to 5 chromosomes present in the biological sample is determined.

20. Method for the quantitative determination of the number n of x predetermined sequence(s) in a biological sample and according to one of the preceding claims comprising the steps:
a) providing a biological sample containing DNA,
b) fragmenting the DNA contained in the biological sample,
c) dividing the sample obtained in the step b) into y subsamples,
d) adding z primer pairs to each of the y subsamples, where to each of the subsamples the same primer pairs are added, and where the individual primer pairs are adapted to amplify, in a PCR, subsequences of the x predetermined sequence(s), said subsequences being different for each primer pair,
e) carrying out a PCR with each of the y subsamples obtained in the step d),
f) determining the number of different amplification products obtained with the PCR reactions in the step e) for the individual subsamples and determining the number of the z subsamples in which identical amplification products have been obtained.

21. Method according to claim 20,
characterized in that
x is a whole number between 1 and 23,
y is a whole number that is greater than or equal to 4, preferably 5 to 20, and
z is a whole number that is greater than or equal to 3·x, preferably 5·x to 25·x and particularly preferably 8·x to 15·x.

22. Method according to claim 21,
characterized in that
the number n of predetermined sequence(s) is determined from the number of the z subsamples in which identical amplification products have been obtained.

23. Method according to claim 20,
characterized in that
the number n per predetermined sequence in the biological sample is 0 to 100, preferably 0 to 10, particularly preferably 0 to 5 and quite particularly preferably 0, 1, 2, 3, or 4.

24. Method according to claim 20,
characterized in that
it is carried out in the framework of an in vitro fertilization (IVF) or in the framework of analysis of fetal cells from maternal blood.

25. Method according to claim 20,
characterized in that
the nucleic acid contained in the sample prepared in the step a) is lysed between the method step a) and the method step b).
